(19) United States
(12) Patent Application Publication
(10) Pub. No.: US 2024/0293636 A1
(43) Pub. Date: Sep. 5, 2024

(54) IMPROVED BYPASS CONDUIT HAVING A PORT

(71) Applicant: SMART RS INC., Ottawa (CA)

(72) Inventor: Francesco FIORENZA, Ottawa (CA)

(73) Assignee: SMART RS INC., Ottawa (CA)

(21) Appl. No.: 18/593,256

(22) Filed: Mar. 1, 2024

Related U.S. Application Data
(60) Provisional application No. 63/449,675, filed on Mar. 3, 2023.

(51) Int. Cl. A61M 16/08

(52) U.S. Cl. CPC A61M 16/0875; A61M 16/0833; A61M 16/208; A61M 2205/3337

(57) ABSTRACT

A bypass conduit is provided comprised of an inlet, an outlet and a bypass. The bypass valve and outlet define a chamber, and a bypass valve is provided within the chamber to allow air to flow from either the inlet to the outlet, or the inlet to the bypass. The chamber is also comprised of an aperture leading to a port, the port configured to either allow gas sampling or medication delivery.

### CROSS REFERENCE TO RELATED APPLICATION

The present application claims priority to U.S. Provisional Application No. 63/449,675, entitled “IMPROVED BYPASS CONDUIT HAVING A PORT” filed on Mar. 3, 2023, the contents of which are incorporated herein by reference in their entirety.

### FIELD

The invention relates to the field of bypass conduits, and more specifically to a respiratory bypass conduit having at least one port.

### BACKGROUND

As is known to those in the respiratory care field, patients on mechanical ventilators are frequently disconnected for a variety of reasons. A circuit disconnection is typically done for a variety of procedures; change filters, circuits, implement adapters for inhaled medications, implement adapters for gas sampling and patient transport. During the procedures, the patient's airflow is interrupted, which allows for possible contaminants or germs to enter the patient, as well as the loss of positive end expiratory pressure (PEEP), which is not a desired effect.

Current bypass conduits allow for the seamless introduction of a secondary ventilation source but importantly, do not allow for gas sampling or medication delivery. As such, additional adapters are required with existing bypass conduits. Unfortunately, additional adapters add additional weight and dead space, which are not desired.

In light of the above, a need exists for an “all in one” bypass conduit to allow for integration of a secondary gas source as well as minimizing ventilator circuit disconnects for the addition of added adapters.

Accordingly, it would be highly beneficial and more efficient if medications could be administered, gas sampling capabilities and the flow of air would be automatically diverted into a bypass conduit allowing for the respiratory system to continue functioning without any interruption. It would also be highly beneficial to have the ability to administer inhaled medications and sample gases no mater which conduit the patient is being ventilated from, which is also extremely convenient during patient transport.

### SUMMARY

In an aspect, the present disclosure provides a bypass conduit for enabling an airflow, comprising: an inlet configured to connect to a ventilator; an outlet operatively engaged with the inlet, the outlet configured to allow the airflow from the inlet to a patient; a bypass connected to the inlet and the outlet, the bypass allowing a bypass of the airflow to the patient; a central chamber positioned at an intersection of the bypass, the outlet and the inlet; a bypass valve positioned in the central chamber, the bypass valve moveable from a first position to a second position; and, at least one port projecting from the central chamber to provide at least one of: gas sampling and medication delivery.

In another aspect, the present disclosure provides a valve for airflow, the valve comprising: a cavity through with air can flow; and, a flow redirector positioned within the cavity, the flow director to redirect the airflow, wherein the flow redirector facilitates the airflow from an outlet to an inlet and impedes the airflow from the inlet to the outlet.

### BRIEF DESCRIPTION OF THE DRAWINGS

The following figures serve to illustrate various embodiments of features of the disclosure. These figures are illustrative and are not intended to be limiting.

FIG. 1 is a perspective view of a bypass conduit according to an embodiment of the present disclosure;

FIG. 2 is a cross-sectional view of the bypass conduit of FIG. 1 taken along the lines FIG. 2, according to an embodiment of the present disclosure;

FIG. 3 is a cross-sectional view of the bypass conduit of FIG. 1 taken along the lines FIG. 3, according to an embodiment of the present disclosure;

FIG. 4 is a perspective view of a bypass conduit according to another embodiment of the present disclosure;

FIG. 5 is a cross-sectional view of the bypass conduit of FIG. 4 taken along the lines FIG. 5, according to an embodiment of the present disclosure;

FIG. 6 is a perspective view of a bypass conduit according to yet another embodiment of the present disclosure;

FIG. 7 is a cross-sectional view of the bypass conduit of FIG. 5 taken along the lines FIG. 7, according to yet another embodiment of the present disclosure;

FIG. 8 is a perspective view of a valve, according to an embodiment of the present disclosure;

FIG. 9 is a cross-sectional view of the valve of FIG. 8, according to an embodiment of the present disclosure;

FIG. 10 is a cross-sectional view of the valve of FIG. 8 with airflow shown, according to an embodiment of the present disclosure;

FIG. 11A is a cross-sectional view of a valve, according to another embodiment of the present disclosure;

FIG. 11B is a cross-sectional view of a valve, according to yet another embodiment of the present disclosure; and,

FIG. 11C is a cross-sectional view of a valve, according to yet another embodiment of the present disclosure.

### DETAILED DESCRIPTION

The following embodiments are merely illustrative and are not intended to be limiting. It will be appreciated that various modifications and/or alterations to the embodiments described herein may be made without departing from the disclosure and any modifications and/or alterations are within the scope of the contemplated disclosure.

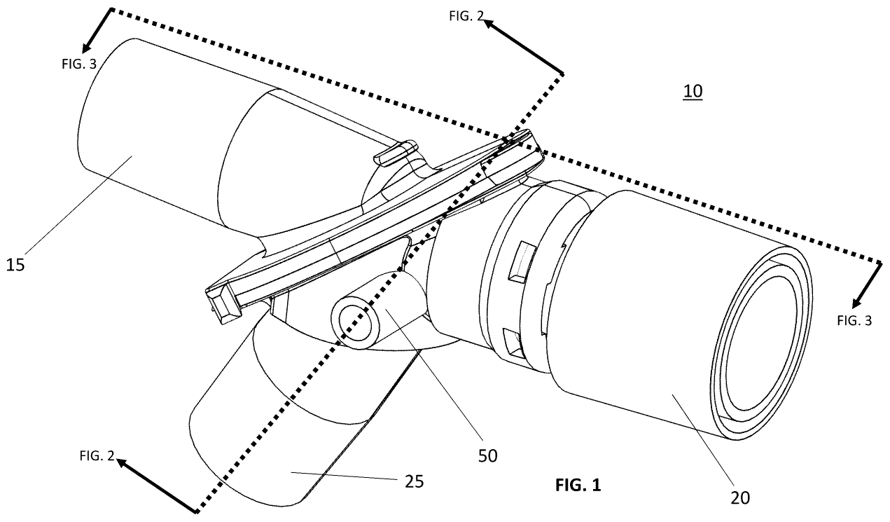

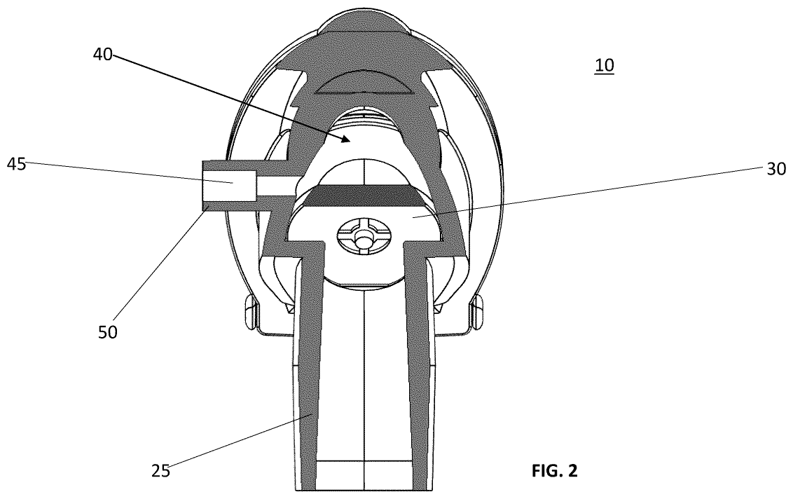

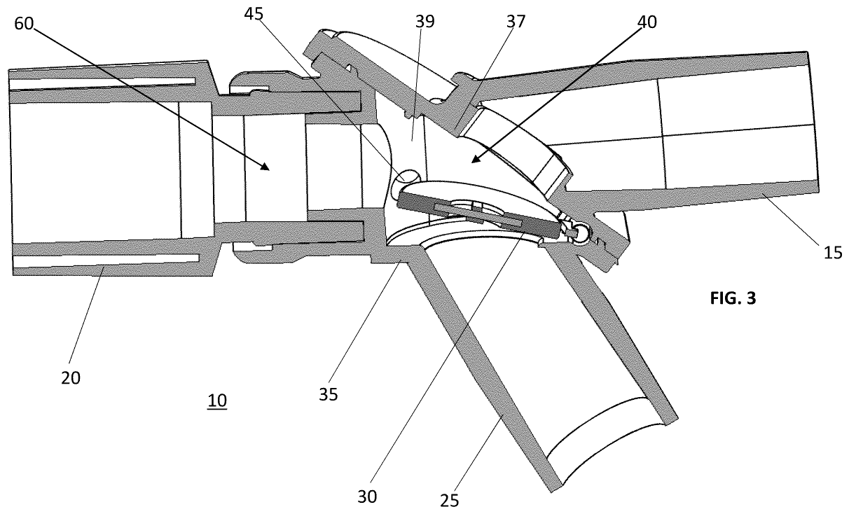

With reference to FIGS. 1, 2 and 3 and according to an embodiment of the present disclosure, a bypass conduit 10 for use in a respiratory bypass conduit is disclosed. The bypass conduit 10 is further comprised of an inlet 15, an outlet 20 and a bypass 25. During operation, air flows from the inlet 15, which is connected to a ventilator, to an outlet 20, which is connected to a patient, thereby allowing the patient to breath. Alternatively, air can flow can be bypassed from the inlet 15 to the bypass 25. The flow of air is determined by the pivotal movement of a bypass valve 30, which pivots from a first position to a second position. The first position is defined as where the bypass valve 30 is sealed against a first wall 35 of the bypass valve 25, thereby allowing air to flow from the inlet 15 to the outlet 20. Meanwhile, the second position is defined as where the bypass valve 30 is sealed against a second wall 37 of the outlet 20, thereby allowing air to flow from the inlet 15 to the outlet 20. Together, the first and second walls 35, 37 cooperate with a third wall 39 to define a central chamber 40 of the bypass conduit 10. The bypass valve 30 moves within this central chamber 40 to pivot from the first position to the second position. The central chamber 40 is comprised of an aperture 45, the aperture 45 leading out to a medicinal port 50. The medicinal port 50 is configured to facilitate medication delivery for the patient. As shown, the aperture 45 leading to the medicinal port 50 is built directly into the central chamber 40. As such, whether the bypass valve 30 is in the first or second position, access to the medicinal port 50 is maintained. Placement of the medicinal port 50 to access the central chamber 40 is important, as it allows for medication delivery irrespective of whether airflow is coming from the bypass 25 or the inlet 15. Optionally, the medicinal port 50 could be positioned in an inlet chamber 60 of the inlet 15. Again, the positioning of the medicinal port 50 in either one of the central chamber 40 or the inlet chamber 60 ensures medication delivery is possible irrespective of whether airflow is coming from the bypass 25 or the inlet 15.

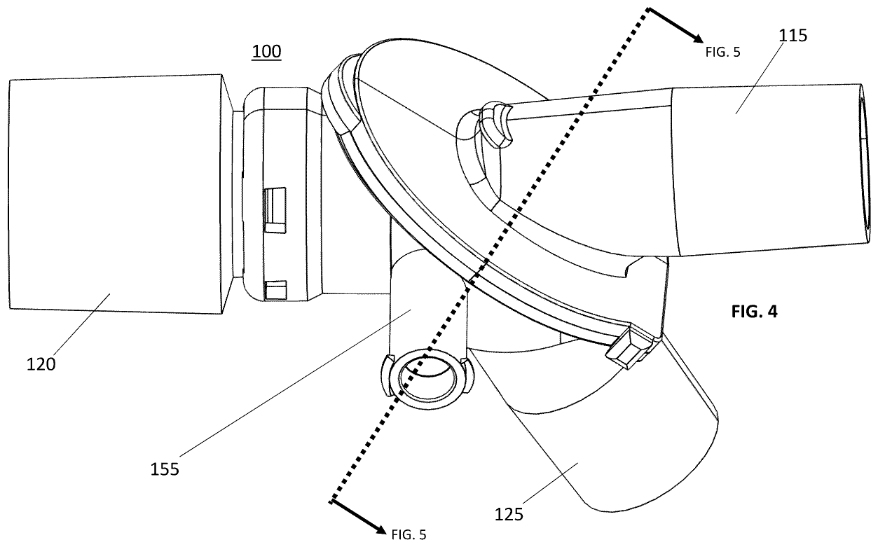

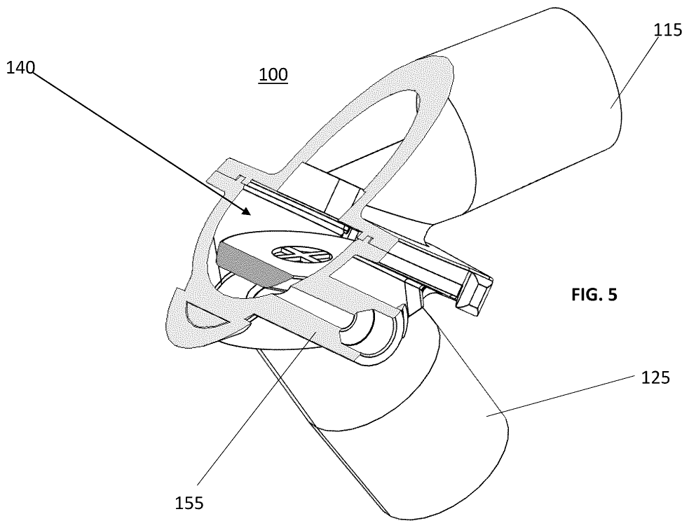

With reference to FIGS. 4 and 5 and according to another embodiment of the present disclosure, a bypass conduit 100 for use in a respiratory bypass conduit is disclosed. The bypass conduit 100 is further comprised of an inlet 115, an outlet 120 and a bypass 125. In this particular embodiment, the medicinal port (not shown) has been replaced by a Luer port 155. In a preferred embodiment, the Luer port 155 is used for gas sampling. In this embodiment, the Luer port 155 is connected to a central chamber 140. Placement of the Luer port 155 to access the central chamber 140 is important, as it allows for gas sampling irrespective of whether airflow is coming from the bypass 125 or the inlet 115. Optionally, the Luer port 155 could be positioned in an inlet chamber (not shown) of the inlet 115. Again, the positioning of the Luer port 155 in either one of the central chamber 140 or the inlet chamber (not shown) ensures gas sampling is possible irrespective of whether airflow is coming from the bypass 125 or the inlet 115.

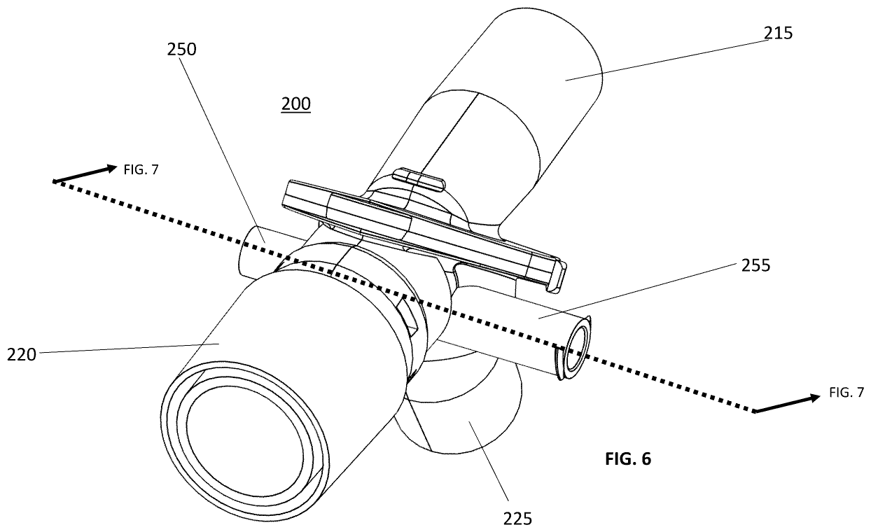

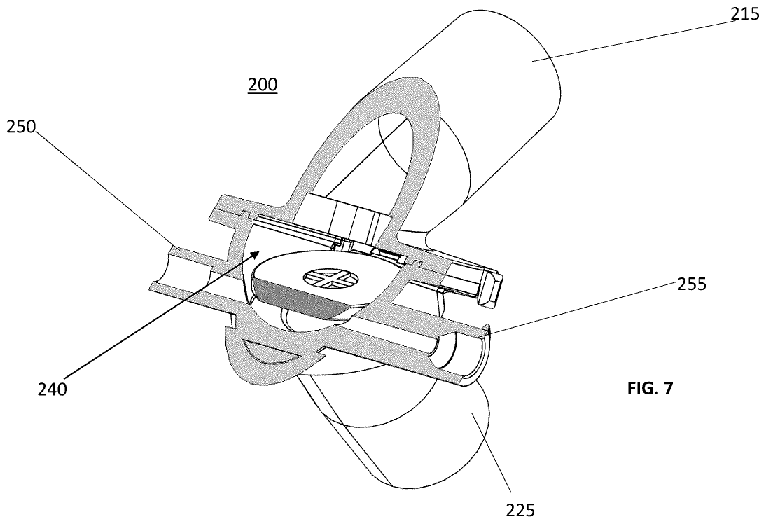

With reference to FIGS. 6 and 7 and according to another embodiment of the present disclosure, a bypass conduit 200 for use in a respiratory bypass conduit is disclosed. The bypass conduit 200 is further comprised of an inlet 215, an outlet 220 and a bypass 225. In this particular embodiment, the bypass conduit 200 is comprised of both a medicinal port 250 and a Luer port 255. In a preferred embodiment, the medicinal port 250 facilitates medication delivery to the patient, whereas the Luer port 255 is used for gas sampling. A worker skilled in the art would appreciate that both the medicinal port 250 and the Luer port 255 are connected to the same central chamber 240 of the bypass conduit 200. Placement of the medicinal port 250 and Luer port 255 to access the central chamber 240 is important, as it allows for medication delivery or gas sampling irrespective of whether airflow is coming from the bypass 225 or the inlet 215. Optionally, the medicinal port 250 and/or the Luer port 255 could be positioned in an inlet chamber (not shown) of the inlet 215, allowing gas sampling or medication delivery irrespective of whether airflow is coming from the bypass 225 or the inlet 215.

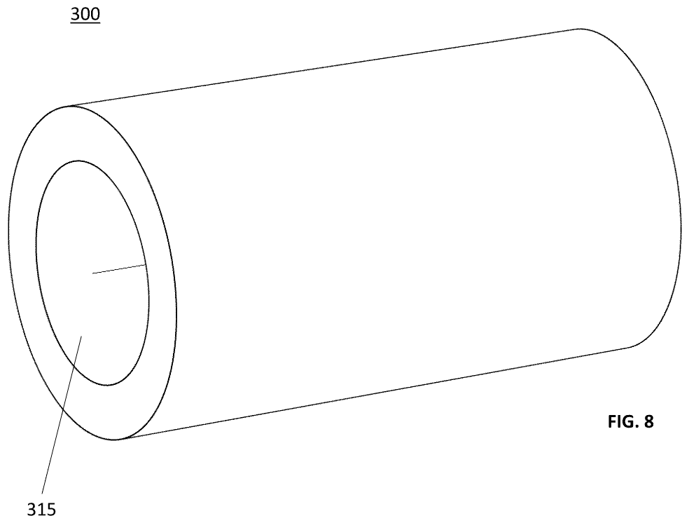

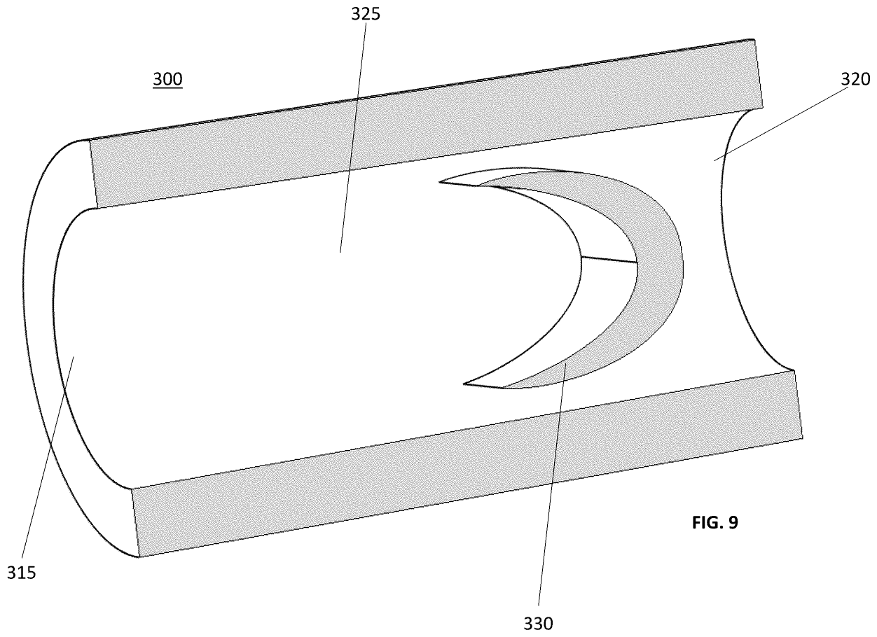

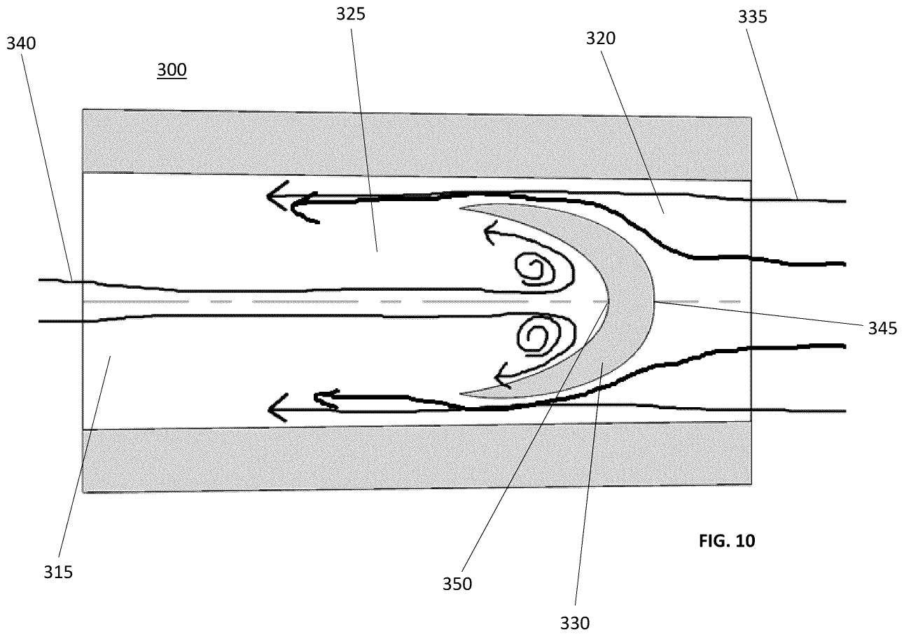

With reference to FIGS. 8, 9 and 10, a valve 300 is shown. In a preferred application, the present valve 300 is a speaking valve 300 for use in tracheotomy, although any application within a ventilator circuit is also possible. The valve 300 is comprised of an inlet 315 configured to connect to a patient (not shown) and an outlet 320 configured to connect to a ventilator (not shown). The valve 300 defines a cavity 325 through which air can flow. A flow redirector 330 is shown positioned within the cavity 325, the flow redirector 330 extending from one side of the cavity 325 to the other, opposed side. In other words, airflow cannot travel above or below the flow redirector 330. A purpose of the flow redirector 330 is to redirect the airflow both from the ventilator (not shown) and the patient (not shown), through both the outlet 320 and inlet 315, respectively. Although all speaking valves as used in the prior art are a one-way valve, the present valve 300 uses a flow redirector 330 to allow for incoming ventilator air, as denoted by ventilator arrows 335 to be redirected to the sides of the flow redirector 300 towards the inlet 315. However, upon exhalation by the patient, as denoted by exhalation arrows 340, the flow redirector 330 creates resistance and turbulent flow to limit and block exhalation from going towards the outlet 320. The flow redirector 330 has a generally U- or boomerang shape. It is understood that the curved outer end 345 of the flow redirector 330 provides a laminar flow and redirects the airflow along arrows 335 towards the inlet 315. Conversely, the curved inner surface 350 of the flow redirector 330 provides turbulence when the airflow denoted by arrows 340 hits the curved inner surface 350 and prevents further airflow to the outlet 320.

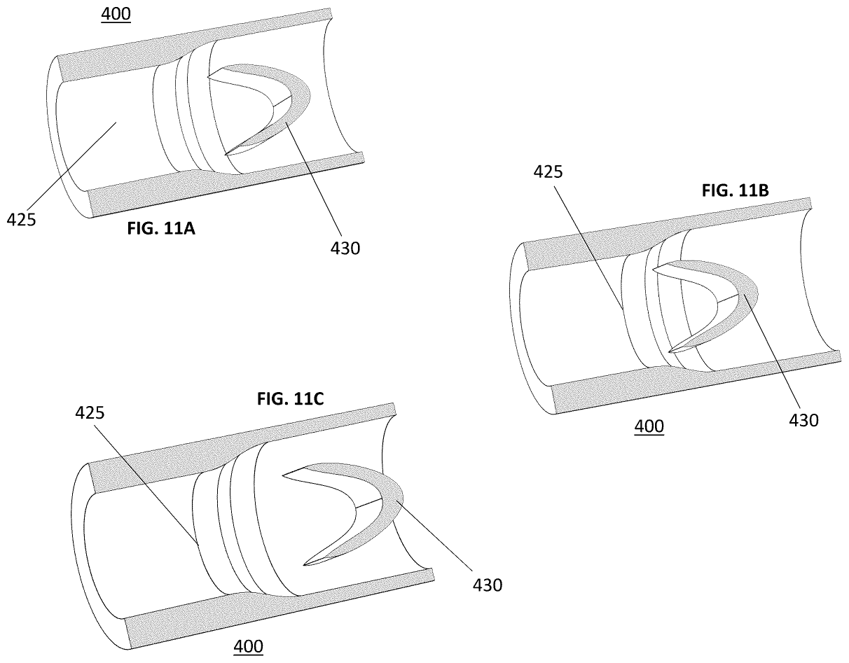

With reference to FIGS. 11A, 11B and 11C, valves 400 are shown, the valves 400 defining a cavity 425 that contain a flow redirector 430. As shown, the internal circumference of the cavities 425 shrinks from the outlet to the inlet, and the flow redirector 430 is positioned at various positions.

Many modifications of the embodiments described herein as well as other embodiments may be evident to a person skilled in the art having the benefit of the teachings presented in the foregoing description and associated drawings. It is understood that these modifications and additional embodiments are captured within the scope of the contemplated disclosure which is not to be limited to the specific embodiment disclosed.

1. A bypass conduit for enabling an airflow, comprising:
an inlet configured to connect to a ventilator;
an outlet operatively engaged with the inlet, the outlet configured to allow the airflow from the inlet to a patient;
a bypass connected to the inlet and the outlet, the bypass allowing a bypass of the airflow to the patient;
a central chamber positioned at an intersection of the bypass, the outlet and the inlet;
a bypass valve positioned in the central chamber, the bypass valve moveable from a first position to a second position; and,
at least one port projecting from the central chamber to provide at least one of: gas sampling and medication delivery.

2. The bypass conduit of claim 1 wherein the first position allows the airflow from the inlet to the outlet and the second position allows the airflow from the inlet to the bypass.

3. The bypass conduit of claim 1 wherein the at least one port is a gas port for the gas sampling.

4. The bypass conduit of claim 1 wherein the at least one port is a medicinal port for the medication delivery.

5. The bypass conduit of claim 1 wherein the at least one port is further comprised of a gas port and a medicinal port.

6. The bypass conduit of claim 1 wherein the at least one port is a tubular member extending from an aperture positioned within the central chamber.

7. A valve for airflow, the valve comprising:
a cavity through with air can flow; and,
a flow redirector positioned within the cavity, the flow redirector to affect the airflow,
wherein the flow redirector facilitates the airflow from an outlet to an inlet and impedes the airflow from the inlet to the outlet.

8. The valve of claim 7 wherein the flow redirector extends from one side of the cavity to a second opposed side.

9. The valve of claim 7 wherein the flow redirector is generally U-shaped.

10. The valve of claim 7 wherein the flow redirector is further comprised of:
a curved outer end to facilitate the airflow from the outlet to the inlet; and,
a curved inner surface to impede the airflow from the inlet to the outlet.

11. The valve of claim 10 wherein the curved outer end creates a laminar flow of air, and the curved inner surface creates a turbulent flow of air.
